(19) United States
(12) Patent Application Publication
(10) Pub. No.: US 2026/0183467 A1
(43) Pub. Date: Jul. 2, 2026

(54) FILTERATION DEVICE FOR USE WITH ENDOSCOPE

(71) Applicant: Gastronaut LLC, Marietta, GA (US)

(72) Inventor: RAJA SHEKHAR REDDY SAPPATI BIYYANI, Marietta, GA (US)

(21) Appl. No.: 19/187,469

(22) Filed: Apr. 23, 2025

Related U.S. Application Data
(60) Provisional application No. 63/637,514, filed on Apr. 23, 2024.

(51) Int. Cl. A61M 1/00; A61B 1/015

(52) U.S. Cl. CPC A61M 1/79; A61B 1/015; A61M 2205/0266

(57) ABSTRACT

The present disclosure relates to a filtration device to be used with an endoscope and a method of using such a filtration device during an endoscopic procedure. The filtration device includes a filter element in a deployed configuration has an outer diameter that is greater than or equal to the diameter of a distal opening of a working channel of an endoscope to provide filtration of the fluid that is suctioned through the working channel of the endoscope. In the deployed configuration, the filter element has pores that are less than 2.5 mm in diameter.

### CROSS-REFERENCE TO RELATED APPLICATIONS

This application claims domestic priority under 35 U.S.C. 119(e) to U.S. Provisional Application Ser. No. 63/637,514, filed Apr. 23, 2024, the disclosures of which is hereby incorporated herein by reference in its entirety.

### COPYRIGHT STATEMENT

A portion of the present disclosure contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the present disclosure, and the applicants make no claim to any trademarks referenced.

### BACKGROUND

### 1. Field of the Invention

The present disclosure is directed to a filtration device for use with an endoscope, a method of making and using the filtration device for use with an endoscope.

### 2. Description of Related Art

An endoscope is a medical instrument that may be used for visualizing the interior of a patient's body. In particular, endoscopes can be used for a variety of different diagnostic and interventional procedures, including an upper endoscopy, a lower endoscopy (i.e., a colonoscopy), a bronchoscopy, a thoracoscopy, a laparoscopy, a video endoscopy, and/or the like.

For example, in one scenario, an endoscope may be inserted by a physician through a patient's esophagus and intubated to a work site. The endoscope may be flexible and may include optical and illuminating features that allow the physician to view the work site. During the procedure, it may become necessary for the physician to evacuate blood clots or other materials in the gastrointestinal tract to clear the field of view. These materials may be removed using one or more aspiration components of the endoscope. In another scenario, an endoscope may be used to guide a feeding tube to the small intestine (e.g., to the jejunum) via the stomach.

Colorectal cancer (CRC), which is characterized by uncontrolled cell growth in the colon or rectum, is the third most prevalent cancer and the fourth deadliest cancer worldwide. Periodic colonoscopies screen for tissue irregularities in the colon and are essential to discover and remove growths known as polyps before they develop into cancer. Clearing the colon of fecal waste prior to a colonoscopy is necessary for proper visualization of the tissue. Patient preparation is deemed to be “inadequate” if the physician is unable to identify tissue abnormalities larger than five millimeters in diameter because of residual solid waste. It is estimated that inadequate prep is observed in more than 25% of colonoscopies; in these situations, doctors have been found to miss between 42-48% of polyps.

In situations where doctors encounter residual solid and liquid fecal matter in a patient's bowel, they will utilize the suction functionality within the colonoscope to try to clear the debris. However, the suction channel has a very small diameter and often becomes clogged with thick liquid and solid waste. In order to clear the clogged material from the suction channel, an endoscopy technician will use a syringe to push water or air from the physician's end of the colonoscope through the suction channel, which is known as a flush. This process can take up to 90 seconds, and, when done repeatedly during a colonoscopy, can extend procedure time by more than 50%. Furthermore, flushes are often ineffective because the waste is pushed directly back into the field of view, or the clogging is so severe that the flush cannot dislodge the clogged material. If the clog is not cleared or the scope becomes damaged due to the high volume of solid material being suctioned, the doctor must remove the endoscope from the patient to perform more extensive cleaning or replace it with a new endoscope. The procedure may also be canceled and rescheduled, which inconveniences both the patient and the clinician and decreases the likelihood that the patient will return for another screening. Overall, physicians performing colonoscopies do not have a way to effectively filter material to prevent suction clogging.

There remains a need for effective filtration to prevent suction clogging during an endoscopic procedure.

### SUMMARY

In a first aspect, provided herein is a filtration device suitable to fit into a working channel of an endoscope. The filtration device comprises a sheath having a proximal end, a distal end, and a distal opening; a proximal operating section connected with the proximal end of the sheath, and a filter assembly enclosed inside the sheath comprising a proximal end connected by a middle section to a filter element positioned close to the distal opening of the sheath. In one embodiment, the proximal operating section of the filtration device is operably connected to the proximal end of the filter assembly and capable of deploying the filter element of the filter assembly to exit the distal opening of the sheath to assume a deployed configuration. In one embodiment, the deployed configuration of the filter element has an outer diameter that is greater than or equal to the diameter of a working channel of an endoscope to provide filtration of the fluid that is suctioned through the working channel of the endoscope. In one embodiment, in the deployed configuration, the filter element comprises pores that are less than 2.5 mm in diameter. In one embodiment, the filter element comprises a three-dimensional cage in the deployed configuration that has a diameter of at least 3 mm. In one embodiment, the filter element comprises an inverted umbrella shaped filter element in the deployed configuration that has a diameter of at least 3 mm. In one embodiment, the endoscope is used for an upper endoscopy, a lower endoscopy or a colonoscopy, a bronchoscopy, a thoracoscopy, a laparoscopy, or a video endoscopy. In one embodiment, the proximal operating section of the filtration device comprises a clip or a biopsy handle to perform the operation of the filter assembly. In one embodiment, the filter element is made from metal wires. In one embodiment, the filter element is made from memory metal wires. In one embodiment, the filter element is pliable. In one embodiment, the pliable filter element fits the contour of the working channel.

In a second aspect, provided herein is a method of using a filtration device for aspiration during an endoscopy procedure using an endoscope, with the endoscope comprises a working channel that has a distal opening at the distal tip. The method comprises the steps of inserting the sheath enclosed with the filter assembly of the filtration device into the working channel of the endoscope until the distal end of the sheath reaches the distal opening of the working channel; using the proximal operating section of the filtration device to deploy the filter element into the deployed configuration to exit the sheath and cover the distal opening of the working channel; and applying suction through the working channel of the endoscope to aspirate fluid through the deployed filter element and into the working channel. In one embodiment, the method further comprises using the proximal operating section of the filtration device to collapse the filter element into a collapsed configuration to retrieve the collapsed filter element back into the sheath and removing the sheath enclosed filter assembly from the working channel of the endoscope. In one embodiment, the method further comprises using the endoscope to examine the aspiration site of the vessel and reposition the distal tip of the endoscope to perform additional aspiration. In one embodiment, the method further comprises using the endoscope to flush the aspiration site with a fluid before the aspiration. In one embodiment, the method further comprises using the endoscope to flush the aspiration site with a fluid after the aspiration to further rinse the aspiration site followed by an additional aspiration. In one embodiment, the method of the aspiration is performed to clear fluid for better visualization in colonoscopy. In one embodiment, the method of the aspiration is performed to clear liquid blood from blood clots in Upper GI bleeding cases. In one embodiment, the method of the aspiration is performed to hold and release a large polyp which is unable to be aspirated out from the working channel of colonoscope in place of a Roth net. In one embodiment, the method of the aspiration is performed to help with removal of food bolus or foreign body obstruction of esophagus. In one embodiment, the method is used for an upper endoscopy, a lower endoscopy or a colonoscopy, a bronchoscopy, a thoracoscopy, a laparoscopy, or a video endoscopy.

In a third aspect, provided herein is a filtration device suitable to fit into a working channel of an endoscope that comprises a sheath having a proximal end, a distal end, and a distal opening, a proximal operating section connected with the proximal end of the sheath, and a pliable filter assembly enclosed inside the sheath comprising a proximal end connected by a middle section to a filter element positioned close to the distal opening of the sheath. In one embodiment, the proximal operating section of the filtration device is operably connected to the proximal end of the filter assembly and capable of deploying the filter element of the filter assembly to exit the distal opening of the sheath to assume a deployed configuration. In one embodiment, the deployed configuration of the filter element has an outer diameter that is greater than or equal to the diameter of a working channel of an endoscope to provide filtration of the fluid that is suctioned through the working channel of the endoscope. In one embodiment, in the deployed configuration, the filter element comprises pores that are less than 2.5 mm in diameter.

In a fourth aspect, provided herein is a method of using the filtration device disclosed herein for aspiration during an endoscopy procedure and the endoscope used comprises a working channel that has a distal opening at the distal tip. The method comprises the steps of inserting the sheath enclosed with the pliable filter assembly of the filtration device into the working channel of the endoscope until the distal end of the sheath reaches the distal opening of the working channel, using the proximal operating section of the filtration device to deploy the pliable filter element into the deployed configuration to exit the sheath to fit the contour of the distal opening of the working channel, and applying suction through the working channel of the endoscope to aspirate fluid through the deployed filter element and into working channel.

### BRIEF DESCRIPTION OF THE DRAWINGS

The drawing figures are not necessarily to scale and certain features may be shown exaggerated in scale or in a somewhat generalized or schematic form in the interest of clarity and conciseness. For more complete understanding of the features and advantages of the present invention, reference is now made to the detailed description along with the accompanying figures, wherein:

FIG. 1 is a schematic diagram of an endoscope in accordance with implementations of various techniques described herein.

FIG. 2 is a schematic diagram illustrating the design and mechanism of a filtration device according to one embodiment of the present disclosure to be used with the endoscope of FIG. 1.

FIG. 3 is a schematic diagram illustrating the details of the filtration device of FIG. 2.

FIG. 3A is a cross-sectional view along the A-A line of portion A of FIG. 3.

FIG. 4 is a schematic diagram illustrating the engineering analysis of FIG. 3.

FIG. 4A is a schematic diagram illustrating the flattened filter assembly.

FIG. 5 is a schematic diagram showing the filtration device of FIG. 2 integrated with the endoscope of FIG. 1.

FIG. 6 is an enlarged view of the distal section of the endoscope of FIG. 1.

FIG. 6a is an enlarged view of the distal section of the endoscope interfaced with the filtration device of FIG. 5 in stage “a” of deployment according to FIG. 2.

FIG. 6b is an enlarged view of the distal section of the endoscope interfaced with the filtration device of FIG. 5 in stage “b” of deployment according to FIG. 2.

FIG. 6c is an enlarged view of the distal section of the endoscope interfaced with the filtration device of FIG. 5 in stage “c” of deployment according to FIG. 2.

FIG. 7 is a schematic diagram illustrating the design and mechanism of a filtration device according to a second embodiment of the present disclosure.

FIG. 7a is an enlarged view of the “a” portion of FIG. 7 showing the filter element in a collapsed configuration.

FIG. 7b is an enlarged view of the “b” portion of FIG. 7 showing the filter element in a semi-open configuration.

FIG. 7c is an enlarged view of the “c” portion of FIG. 7 showing the filter element in a deployed configuration.

FIG. 8 is a schematic diagram showing the filtration device of FIG. 7 integrated with the endoscope of FIG. 1.

FIG. 8A is an enlarged view of the distal section of the endoscope of FIG. 8 in stage A of deployment showing the filter element in a collapsed configuration.

FIG. 8B is an enlarged view of the distal section of the endoscope of FIG. 8 in stage B of deployment showing the filter element in a semi open configuration.

FIG. 8C is an enlarged view of the distal section of the endoscope of FIG. 8 in stage C of deployment showing the filter element in a deployed configuration.

FIG. 9 is a schematic diagram illustrating the design and mechanism of a filtration device according to a third embodiment of the present disclosure.

FIG. 9a is an enlarged view of the “a” portion of FIG. 9 showing the filter element in a collapsed configuration.

FIG. 9b is an enlarged view of the “b” portion of FIG. 9 showing the filter in a semi-open configuration.

FIG. 9c is an enlarged view of the “c” portion of FIG. 9 showing the filter in a deployed configuration.

FIG. 10 is a schematic diagram showing the filtration device of FIG. 9 integrated with the endoscope of FIG. 1.

FIG. 10A is an enlarged view of the distal section of the endoscope of FIG. 10 in stage A of deployment showing the filter element in a collapsed configuration.

FIG. 10B is an enlarged view of the distal section of the endoscope of FIG. 10 in stage B of deployment showing the filter element in a semi open configuration.

FIG. 10C is an enlarged view of the distal section of the endoscope of FIG. 10 in stage C of deployment showing the filter element in a deployed configuration.

FIG. 11 is a schematic diagram showing one embodiment of the filtration device of FIG. 7 integrated with the endoscope of FIG. 1 where the filter element stays inside the working channel of the endoscope.

FIG. 11A is an enlarged view of the distal section of the endoscope of FIG. 11 in stage A of deployment showing the filter element in a collapsed configuration inside the working channel of the endoscope.

FIG. 11B is an enlarged view of the distal section of the endoscope of FIG. 11 in stage B of deployment showing the filter element in a semi open configuration inside the working channel of the endoscope.

FIG. 11C is an enlarged view of the distal section of the endoscope of FIG. 11 in stage C of deployment showing the filter element in a deployed configuration inside the working channel of the endoscope.

FIG. 12 is a schematic diagram showing one embodiment of the filtration device of FIG. 9 integrated with the endoscope of FIG. 1 where the filter element stays inside the working channel of the endoscope.

FIG. 12A is an enlarged view of the distal section of the endoscope of FIG. 12 in stage A of deployment showing the filter element in a collapsed configuration inside the working channel of the endoscope.

FIG. 12B is an enlarged view of the distal section of the endoscope of FIG. 12 in stage B of deployment showing the filter element in a semi open configuration inside the working channel of the endoscope.

FIG. 12C is an enlarged view of the distal section of the endoscope of FIG. 12 in stage C of deployment showing the filter element in a deployed configuration inside the working channel of the endoscope.

FIG. 13 is a flow chart illustrating the steps in a method of using the filtration device disclosed herein.

FIG. 14 is a flow chart illustrating the steps in a method of using the filtration device disclosed herein, where the filter element stays inside the distal opening of the working channel of the endoscope as illustrated in FIGS. 11, 11A-C, 12, and 12A-C.

### DETAILED DESCRIPTION

### Definitions

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a composition that “comprises,” “has,” “includes” or “contains” one or more elements possesses those one or more elements but is not limited to possessing only those elements. Likewise, a method that “comprises,” “has,” “includes” or “contains” one or more steps possesses those one or more steps but is not limited to possessing only those one or more steps.

The filtration device disclosed herein is also referred to as EndoSieve or EndoMesh throughout the present disclosure.

Any embodiment of any of the apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

The present disclosure may be understood more readily by reference to the following detailed description of embodiments and to the Figures and their previous and following description.

Various implementations of devices for use with an endoscope are described herein.

Physicians may use endoscopes during minimally invasive procedures to visualize a patient's anatomy, diagnose various conditions, and/or deliver devices to a treatment site. Such devices may include forceps, scissors, brushes, snares, baskets, and/or the like. As noted above, endoscopes can be used for a variety of different diagnostic and interventional procedures, including an upper endoscopy, a lower endoscopy (i.e., a colonoscopy), a bronchoscopy, a thoracoscopy, a laparoscopy, a video endoscopy, and/or the like.

For example, in colonoscopy where doctors encounter residual solid and liquid fecal matter in a patient's bowel, to improve patient outcomes and physician frustration, a filtration device a.k. a EndoSieve or EndoMesh disclosed herein can be inserted through the suction or working channel of a colonoscope to prevent solid material from accumulating in the channel and clogging it. The filtration device deploys a filter element that covers the channel and forms a tight seal when suction is applied to block solid material that are larger than a predetermined size, such as 2 mm, to prevent clogging. Once filtering is complete, the device can collapse to fit back through the suction channel. This allows the physician to insert and remove the device as necessary without having to withdraw the entire colonoscope, reducing the time of the colonoscopy procedure in these cases.

More than 15 million colonoscopies are performed annually in the United States alone, with poor prep occurring in around 25% of procedures (3.75M)¹⁷. The Roth Net is another colonoscopic tool that does not address the issue of suction clogging during colonoscopies, but it serves as a reasonable comparison for the cost of a potential device. The average sales price of a Roth Net is $85.40, meaning the total addressable market for the clinical problem is $85.40*15M cases or $1.28B/year. Assuming that a future filtration device would be utilized only during cases where poor bowel prep is observed and one device is used per patient, the serviceable available market is $85.40*3.75M or $320.25M/year¹⁸.

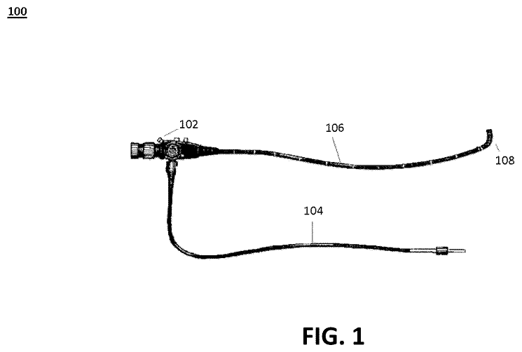

Referring to FIG. 1, a schematic diagram of an endoscope 100 in accordance with implementations of various techniques described herein is shown. The endoscope 100 may include a proximal handle 102, a cord 104, and an insertion tube 106 with a distal tip 108. Those skilled in the art will understand that other configurations for an endoscope may also be used with respect to the implementations described herein.

The proximal handle 102 may be coupled to both the cord 104 and the insertion tube 106. Though not shown, the proximal handle 102 may include an eyepiece, one or more valves for wires and instrument, and any other component known to those skilled in the art. Such valves may include a suction valve, a biopsy valve, a water valve, an air valve, and/or the like. In particular, the proximal handle 102 may be used to control operation of the endoscope 100, particularly the insertion tube 106 and its distal tip 108. The cord 104 may include one or more components and/or connectors that are configured to supply light, imaging, air, water, and/or the like to the insertion tube 106. For example, the cord 104 may be coupled to a positive air pressure source, a water tank, a negative air pressure source, and/or the like.

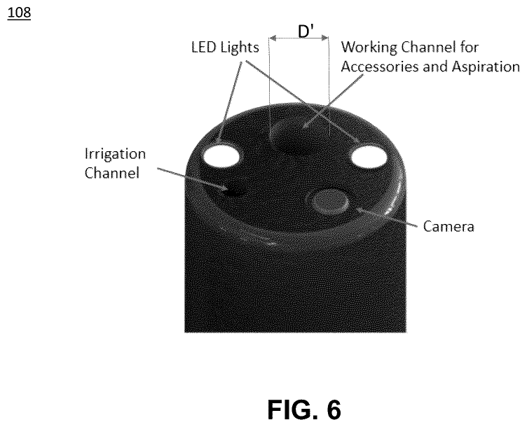

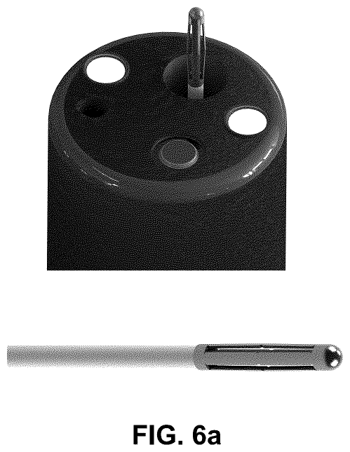

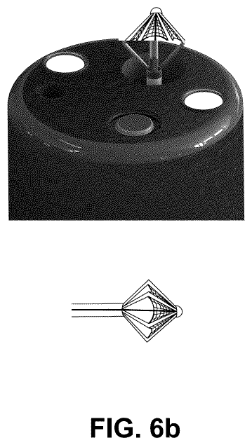

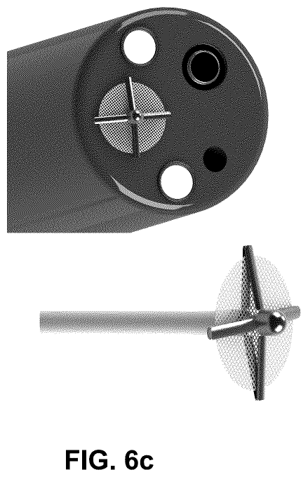

The insertion tube 106 and the distal tip 108 may be configured to be inserted into a patient's anatomy during one of the procedures mentioned above. The insertion tube 106 may be a long, thin tube that is configured to be flexible or rigid. The insertion tube 106 may further include one or more channels disposed therein. For example, the insertion tube 106 may include one or more irrigation channels (e.g., an air channel, a water channel, etc.), a working channel, and an illumination system (e.g., a light source, a camera lens, etc.), as shown in FIG. 6. The working channel may also be referred to as the biopsy/suction channel of the endoscope. In some implementations, the working channel may be used for suction, to deliver one or more devices, and/or the like at a work site in the patient.

The distal tip 108 may include one or more assemblies related to the illumination system, such as the light source and the camera lens. The distal tip 108 may also include openings for the one or more irrigation channels and the working channel. FIG. 6 illustrates a schematic diagram of a front view of the distal tip 108 of the endoscope 100 in accordance with implementations of various techniques described herein. As shown, the distal tip 108 may include an opening for the working channel, openings for the one or more irrigation channels, and a light source and camera lens for use with the illumination system. Those skilled in the art will understand that other configurations for a distal tip of an endoscope may also be used with respect to the implementations described herein.

Using the proximal handle 102 (with the associated eyepiece, one or more valves, etc.), cable 104, and insertion tube 106 (with the associated one or more irrigation channels, working channel, and illumination system), a physician may perform a number of tasks. For example, the working channel, via its opening at the distal tip 108, may be used to pass one or more devices through for performing diagnostic or therapeutic procedures within the field of view of the endoscope 100. In another example, by activating a suction valve of the proximal handle 102, the physician may aspirate soiled material, secretions, water, blood, other fluids, air, and/or the like from a work site of the patient via the opening of the working channel. In yet another example, the endoscope 100, via the opening of its working channel, may employ a device to guide a feeding tube to the small intestine (e.g., to the jejunum) via the stomach.

As noted above, in some scenarios, a physician may use an endoscope to aspirate soiled material, secretions, water, blood, other fluids, air, and/or the like from a work site of the patient via a distal tip of the endoscope, such as during an upper or lower endoscopy (i.e., a colonoscopy). For example, during a colonoscopy, a physician may encounter pools of liquid and semi-solid material inside of a patient's colon, which may occur even for patients with good, fair, or adequately prepared colons. In such an example, a visualization of the patient's mucosa may be limited, so the working channel may be used to aspirate the liquid and semi-solid material.

However, in some instances, when aspirating during an endoscopy, the working channel (i.e., the biopsy/suction channel) may become clogged with seeds or solid fecal material, thereby interrupting the suction. In such instances, a change of the endoscope may be required during the procedure. Changing the endoscope may lead to a prolonged procedure time and may lead to dissatisfaction among the physician, members of the endoscopy team, and/or the patient. Moreover, in such instances, the endoscope may need to be sent for repair if seeds or solid fecal material become lodged within the working channel.

In other scenarios, a physician may use an endoscope perform a polypectomy. In some instances, the physician may be unable to aspirate the polyp material if the material is relatively large. For such instances, a net device may be used to retrieve the polyp material, followed by a withdrawal of the endoscope. However, if additional polyp material is noted during the withdrawal, the physician may be unable to perform any therapeutic maneuvers on the additional material due to the presence of net device. In particular, the endoscope may have to be withdrawn completely and reintroduced to work on the remaining polyp material. Once again, this may lead to a prolonged procedure time and may lead to dissatisfaction among the physician, members of the endoscopy team, and/or the patient.

In view of the above, various implementations for a filtration device for use with an endoscope are described herein. In one or more implementations, the filtration device may be configured to be passed through a working channel (i.e., a biopsy/suction channel) of an endoscope, and may be used during an upper or lower endoscopy. In one embodiment, the filtration device may include a net configured to open in a forward direction in a similar manner as an opening of an umbrella.

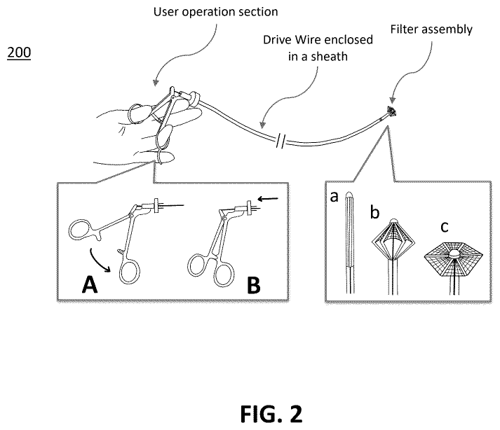

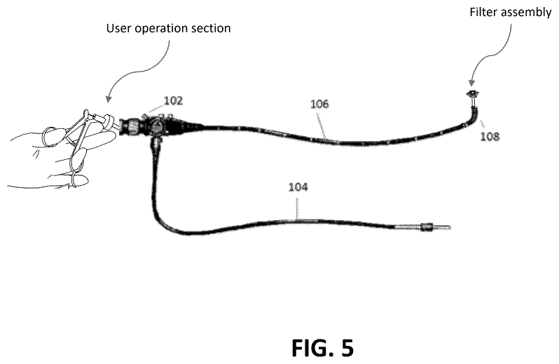

FIG. 5 illustrates a schematic diagram of the endoscope of FIG. 1 interfaced with the filtration device of FIG. 2 in accordance with implementations of various techniques described herein. As shown, the filtering assembly of the filtration device may be in an open configuration and extending outwardly from an opening of a working channel at the distal tip 108 of the endoscope. In some implementations, the filtration device may include two bores of plastic tubing, which may be configured to not inhibit suction power of the endoscope. In another implementation, the filtration device comprises a filter assembly extending from the distal tip 108. In one such implementation, the filter assembly may be similar to various net devices known in the art that are used with endoscopes.

In another implementation, when in an open configuration and extending forwardly (like an umbrella), the filter assembly may be configured to cover the opening of the working channel, as show in FIG. 6c. In such an implementation, the filtering assembly may resemble a disc from a particular viewpoint when the filtering assembly is at its maximum diameter. In one implementation, the filter assembly of FIG. 2 may have a size of 6 Fr in the French Gauge system, though other sizes may be used. In such an implementation, the mesh-like material of the filter assembly may not allow material with a size larger than 6 Fr to be suctioned via the working channel of the endoscope. The positions a, b, c of the filter assembly according to FIG. 2 in the context of the working channel of the enlarged tip section of the endoscope shown in FIGS. 6a, 6b, and 6c, respectively.

In operation, the filtration device disclosed herein may help to separate liquids from solid, which may enhance visualization and/or prevent clogging of the working channel during an endoscopy. In particular, by suctioning the liquids out from the work site using the filtration device disclosed herein, the mucosa could be better examined. Moreover, the filtration device may be used to move around any remnant solid material.

In one implementation, the filtration device may be configured to help separate liquids from solids when aspirating a colon using an endoscope, thereby protecting the working channel (e.g., for a poorly prepared colon). In particular, the filtration device may help to prevent the aspiration of solid fecal material and/or seeds during an endoscopy, thereby protecting the working channel of the endoscope.

In another implementation, the filtration device may also be configured to protect the working channel during an endoscopic aspiration of retained liquid food or blood around a large blood clot while working in the stomach. In particular, during an upper endoscopy, the filtration device may be useful in separating the residual liquid from solid food material and liquid blood from around blood clots.

In yet another implementation, the filtration device herein, with its filter assembly in an open configuration and extending forwardly (like an umbrella), may be configured to hold relatively large polyp material after a polypectomy. In such an implementation, the filtration device may be configured to release the polyp material when and where needed, such as if another polyp is seen and need to be worked upon. In particular, the polyp material can be grabbed as well as released as needed to work on other polyps, thereby reducing procedure time significantly and increasing the efficacy of the endoscopic procedure.

In another implementation, the filtration device may also help with food impaction and/or foreign body removal. In particular, the filtration device can be used to remove an impacted foreign body and/or food bolus from the esophagus. In a further implementation, once the aspirations described above have finished, the filtration device may be closed (e.g., like closing an umbrella) without particulate matter being lodged in the working channel. The filtration device may also be removed with relative ease from the endoscope, such that it can be used when needed in another area of the patient.

FIG. 2 is a schematic diagram illustrating the design and mechanisms for flat umbrella concept with a scissor handle, according to one embodiment of the present disclosure. The design, shown in FIG. 2, uses a scissor-shaped handle that is operated by the physician's thumb and middle or ring finger. When the two pieces of the handle are “closed,” or hinged together, the drive wire is pulled backwards. At the distal end of the device, there are thin folding arms that are bent when the drive wire pulls on the end cap. As these arms collapse and bend, it allows a filter to unfold between them into a flat “plate” shape. This flattened shape fits over the suction channel and provides the highest level of contact with the outside of the device, forming a tight seal when suction is applied. Furthermore, the scissor handle has two rigid arms that lock together when the two handle halves are closed (position A), allowing the filter to remain in the deployed position (position c). When the handle is unlocked and pulled apart (position B), the drive wire is pushed forward, straightening the arms of the filter back into a tubular shape (position a). When the scissor handle is in a transition phase between position A and position B, the drive wire is halfway pushed forward and the filter assembly is in a half-deployed position (position b). In some embodiments, a clip or biopsy handle could be used as the proximal operating section of the filtration device to deploy and maneuver the distal filter element.

The filtration device disclosed herein, while intended for colonoscopies, could be applied for similar filtering purposes in any type of endoscopic procedure. An example is the filtering of blood clots during upper endoscopies⁷.

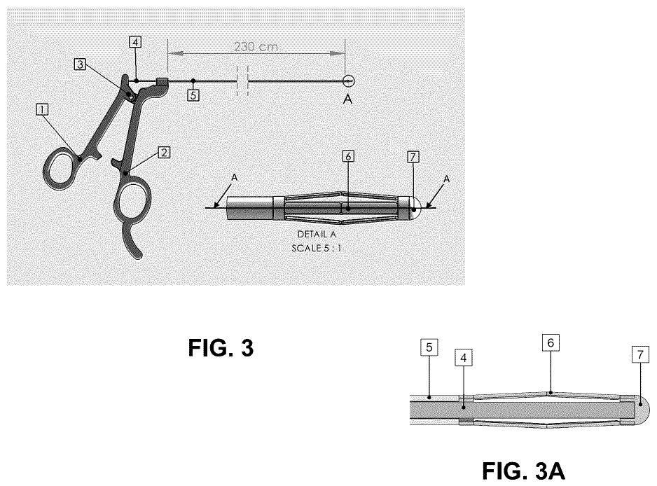

Referring to FIG. 3, the filter device comprises a proximal operating section that comprises handle arms (FIG. 3: items 1 and 2). In one embodiment, these handle arms are injection molded using ABS plastic, the hinge (FIG. 3: item 3) is achieved using a screw and washer, or an injection-molded plastic hinge pin. In one embodiment, the drive wire (FIG. 3: item 4) is made of a stainless steel or nitinol wire. In one embodiment, the outer tube (FIG. 3: item 5) is made of Polytetrafluoroethylene (PTFE). In one embodiment, the folding arms (FIG. 3: item 6) are laser cut from a hollow nitinol tube. In one embodiment, the end cap (FIG. 3: item 7) is an injection molded from ABS plastic or machined from nitinol. In one embodiment, the filter mesh is made from polyester.

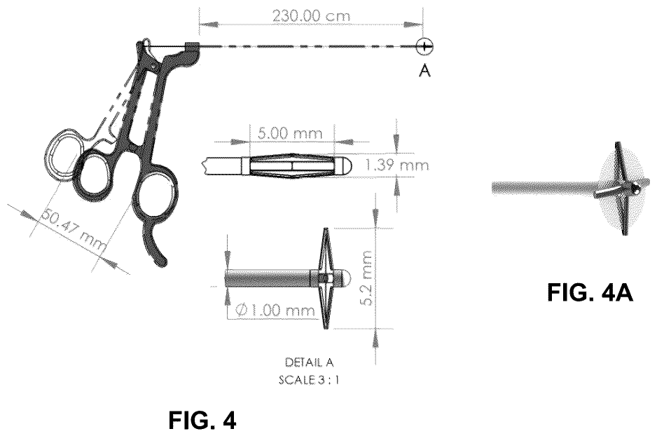

Referring to FIG. 4, an engineering analysis of the filtration device of FIG. 2 is shown. Specifically, the dimensions of the filtration device have been calculated to fit two critical parameters. In one embodiment, when the handle is hinged 50.47 mm from the open position to the closed position, the filter end effector will deploy to a radius of 5.2 mm, which is large enough to cover the largest suction channel found in colonoscopes (4.2 mm) with 0.5 mm of overlap²⁵. To determine the ideal distance of handle hinging, 5 subjects were tested for their maximum range of motion using the handle design, and the smallest maximum distance achieved by a subject was found to be 58 mm. Therefore, the motion required for operation is within that range and will be comfortably achievable for a wide range of physicians. When the bending arms of the filtering assembly are flattened, the filter assembly will fully deploy into a flat shape, as shown in FIG. 4A.

When the handle is opened, the bending arms condense to a diameter of approximately 1.39 mm, which is small enough to fit back through the smallest suction channel found in colonoscopes (2.8 mm)²⁵.

In one embodiment, the filtration device disclosed herein is used to clear fluid for better visualization in colonoscopy. In one embodiment, the filtration device disclosed herein is used to clear liquid blood from blood clots in Upper GI bleeding cases. In one embodiment, the filtration device disclosed herein is used to hold and release a large polyp which is unable to be aspirated out from the working channel of colonoscope in place of a Roth net. In one embodiment, the filtration device disclosed herein is used to help with removal of food bolus or foreign body obstruction of esophagus.

The suction or working channel of an endoscope varies in diameters. For example, the suction or working channel of a standard upper endoscope is 2.8 mm. The suction or working channel of a therapeutic upper endoscope is 3.8-4.2 mm. The suction or working channel of a standard colonoscope 3.7 mm. The suction or working channel of a pediatric colonoscope is 3.2-3.8 mm.

To prevent clogging, the present of pores in the filter element in some embodiments of the present disclosure will allow fluid containing smaller particulates to pass through the filter element while preventing larger particulates from entering and clogging the working channel of the endoscope. In one embodiment, the filter element comprises pores that are less than 2.5 mm in diameter, for example, between 2.4-2.5 mm, 2.2-2.4 mm, 2.0-2.2 mm, 1.8-2.0 mm, 1.6-1.8 mm, 1.4-1.6 mm, 1.2-1.4 mm, 1.0-1.2 mm, 0.8-1.0 mm, 0.6-0.8 mm, 0.4-0.6 mm, or 0.2-0.4 mm in diameter. The pores can be of same, similar, different, or a combination of same, similar, or different sizes. In some embodiments, a combination of pore sizes is used in the filter element of the filtration device disclosed herein.

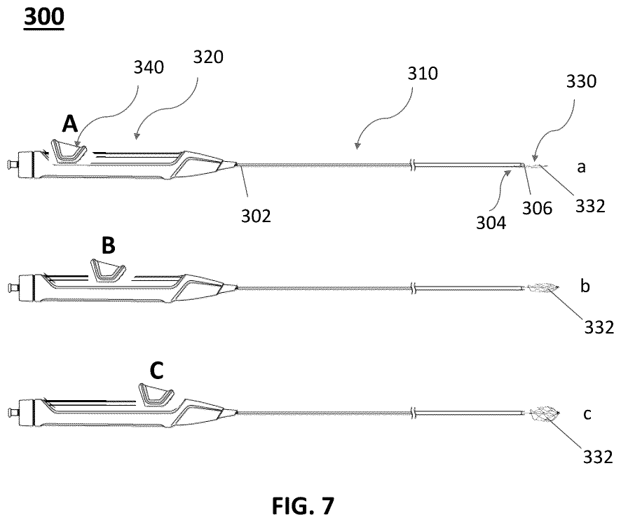

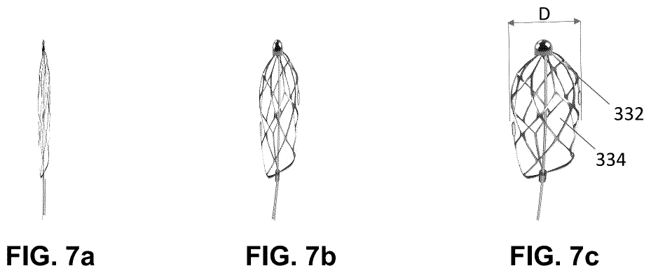

Referring to FIG. 7, a schematic diagram illustrating the design and mechanism of a filtration device according to a second embodiment of the present disclosure is shown. 1. The filtration device 300 is designed to be suitable to fit into a working channel of an endoscope. The filtration device 300 comprises a sheath 310 having a proximal end 302, a distal end 304, and a distal opening 306. The filtration device 300 also comprises a proximal operating section or handle 320 connected with the proximal end 302 of the sheath. A filter assembly 330 of the filtration device 300 is enclosed inside the sheath 310 comprising a proximal end connected by a middle section to a filter element 332 positioned close to the distal opening 306 of the sheath 310. The proximal operating section 320 of the filtration device 300 is operably connected to the proximal end of the filter assembly and capable of deploying the filter element of the filter assembly to exit the distal opening of the sheath to assume a deployed configuration. In one embodiment, the filter element deployment is performed by sliding a slider button 340. As shown in FIG. 7, the slider button 340 could assume positions A, B, and C that corresponds to filter element configurations a, b, and c, which are collapsed, semi-open, and deployed configurations of the filter element. Specifically, FIG. 7a illustrates an enlarged view of the “a” portion of FIG. 7 showing the filter element in a collapsed configuration. FIG. 7b illustrates an enlarged view of the “b” portion of FIG. 7 showing the filter element in a semi-open configuration. FIG. 7c illustrates an enlarged view of the “c” portion of FIG. 7 showing the filter element in a deployed configuration. As shown in FIG. 7c, the deployed configuration of the filter element 332 has an outer diameter D that is greater than or equal to the diameter D′ (shown in FIG. 6) of a working channel of an endoscope to provide filtration of the fluid that is suctioned through the working channel of the endoscope. In one embodiment, the working channel of the endoscope has a diameter between 2.5 mm to 4.5 mm. In one embodiment, the filter element 332 comprises pores 334 that are less than 2.8 mm in diameter, for example, between 2.6-2.8 mm, 2.4-2.6 mm, 2.2-2.4 mm, 2.0-2.2 mm, 1.8-2.0 mm, 1.6-1.8 mm, 1.4-1.6 mm, 1.2-1.4 mm, 1.0-1.2 mm, 0.8-1.0 mm, 0.6-0.8 mm, 0.4-0.6 mm, or 0.2-0.4 mm in diameter. The pores can be of same, similar, different, or a combination of same, similar, or different sizes. In some embodiments, a combination of pore sizes is used in the filter element of the filtration device disclosed herein.

To prevent clogging, the present of pores in the filter element in some embodiments of the present disclosure will allow fluid containing smaller particulates to pass through the filter element while preventing larger particulates from entering and clogging the working channel of the endoscope. In one embodiment, the filter element comprises pores that are less than 2.5 mm in diameter, for example, between 2.4-2.5 mm, 2.2-2.4 mm, 2.0-2.2 mm, 1.8-2.0 mm, 1.6-1.8 mm, 1.4-1.6 mm, 1.2-1.4 mm, 1.0-1.2 mm, 0.8-1.0 mm, 0.6-0.8 mm, 0.4-0.6 mm, or 0.2-0.4 mm in diameter. The pores can be of same, similar, different, or a combination of same, similar, or different sizes. In some embodiments, a combination of pore sizes is used in the filter element of the filtration device disclosed herein.

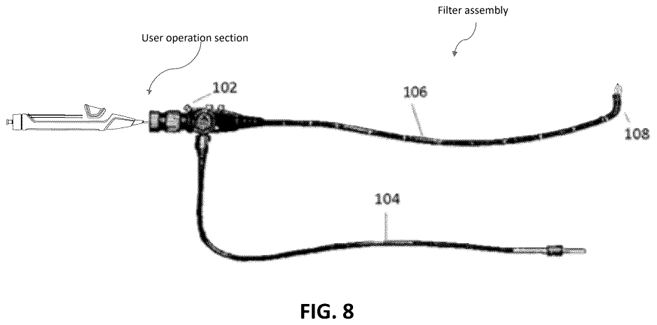

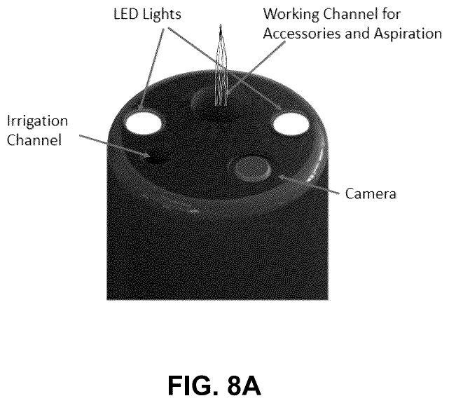

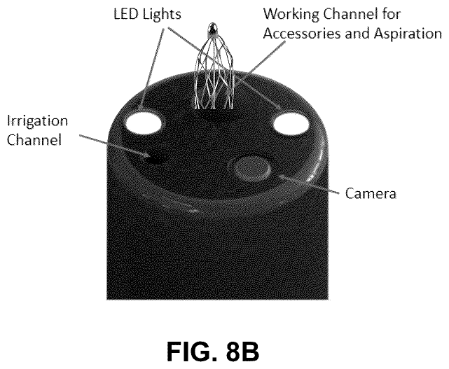

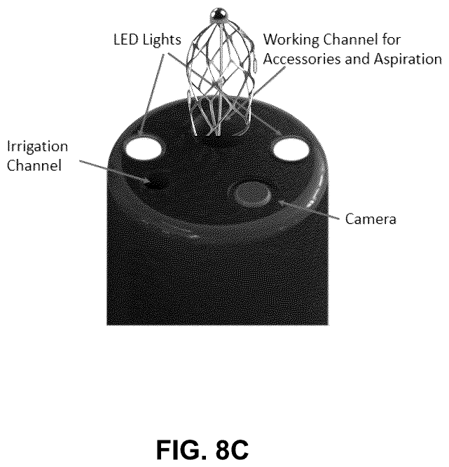

Referring to FIG. 8, the filtration device of FIG. 7 is shown to be integrated with the endoscope of FIG. 1, with the filter element in a deployed configuration covering the distal opening of the working channel. Specifically, the collapsed, semi-open, and deployed configurations of the filter element are shown in the enlarged view of the distal section of the endoscope of FIG. 8 in stages A, B, and C of deployment in FIGS. 8A, 8B, and 8C respectively.

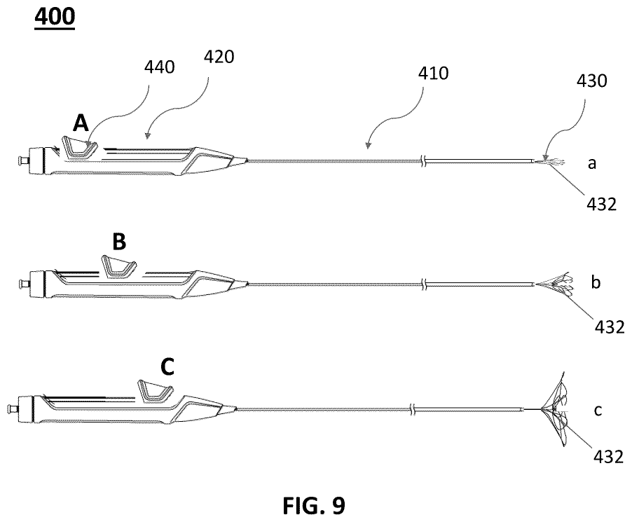

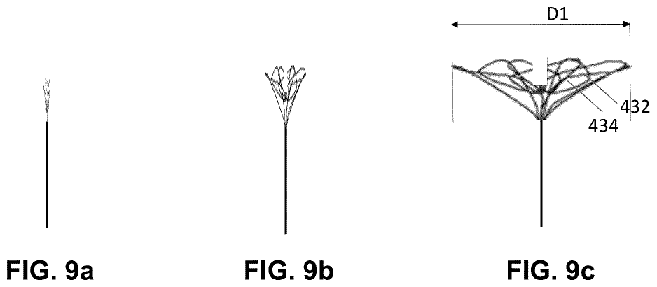

A third embodiment of the filtration device 400 of the present disclosure is illustrated in FIG. 9, showing the design and mechanism of a filter assembly 430 comprising a filter element 432 having an inverted umbrella basket profile in the deployed configuration. In one embodiment, the filter element deployment is performed by sliding a slider button 440. As shown in FIG. 9, the slider button 440 could assume positions A, B, and C that corresponds to filter element configurations a, b, and c, which are collapsed, semi-open, and deployed configurations of the filter element 432. Specifically, FIG. 9a illustrates an enlarged view of the “a” portion of FIG. 9 showing the filter element 432 in a collapsed configuration. FIG. 9b illustrates an enlarged view of the “b” portion of FIG. 9 showing the filter element 432 in a semi-open configuration. FIG. 9c illustrates an enlarged view of the “c” portion of FIG. 9 showing the filter element in a deployed configuration. As shown in FIG. 9c, the deployed configuration of the filter element 432 has an outer diameter D1 that is greater than or equal to the diameter D′ (shown in FIG. 6) of a working channel of an endoscope to provide filtration of the fluid that is suctioned through the working channel of the endoscope. In one embodiment, the working channel of the endoscope has a diameter between 2.5 mm to 4.5 mm. In one embodiment, the filter element 432 comprises pores 434 that are less than 2.8 mm in diameter, for example, between 2.6-2.8 mm, 2.4-2.6 mm, 2.2-2.4 mm, 2.0-2.2 mm, 1.8-2.0 mm, 1.6-1.8 mm, 1.4-1.6 mm, 1.2-1.4 mm, 1.0-1.2 mm, 0.8-1.0 mm, 0.6-0.8 mm, 0.4-0.6 mm, or 0.2-0.4 mm in diameter. The pores can be of same, similar, different, or a combination of same, similar, or different sizes.

To prevent clogging, the present of pores in the filter element in some embodiments of the present disclosure will allow fluid containing smaller particulates to pass through the filter element while preventing larger particulates from entering and clogging the working channel of the endoscope. In one embodiment, the filter element comprises pores that are less than 2.5 mm in diameter, for example, between 2.4-2.5 mm, 2.2-2.4 mm, 2.0-2.2 mm, 1.8-2.0 mm, 1.6-1.8 mm, 1.4-1.6 mm, 1.2-1.4 mm, 1.0-1.2 mm, 0.8-1.0 mm, 0.6-0.8 mm, 0.4-0.6 mm, or 0.2-0.4 mm in diameter. The pores can be of same, similar, different, or a combination of same, similar, or different sizes. In some embodiments, a combination of pore sizes is used in the filter element of the filtration device disclosed herein.

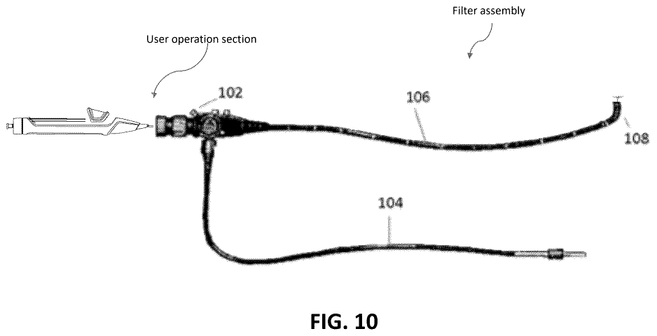

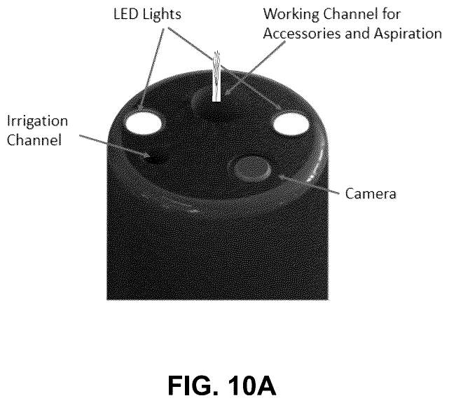

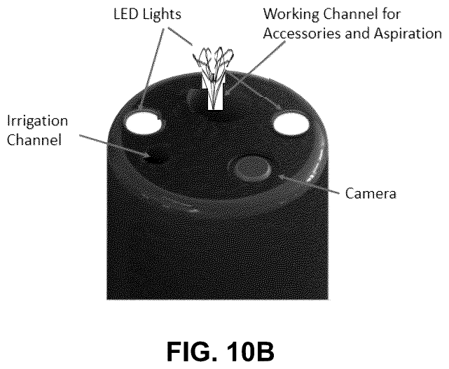

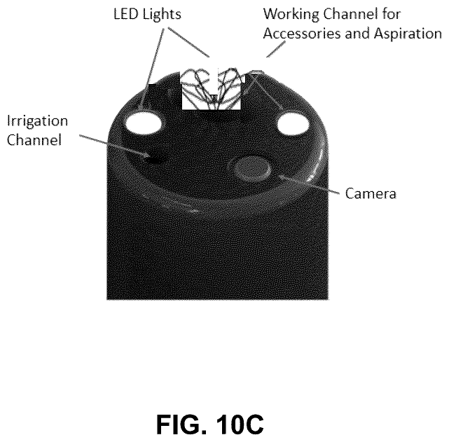

Referring to FIG. 10, the filtration device of FIG. 9 is shown to be integrated with the endoscope of FIG. 1, with the filter element in a deployed configuration covering the distal opening of the working channel. Specifically, the collapsed, semi-open, and deployed configurations of the filter element are shown in the enlarged view of the distal section of the endoscope of FIG. 10 in stages A, B, and C of deployment in FIGS. 10A, 10B, and 10C respectively.

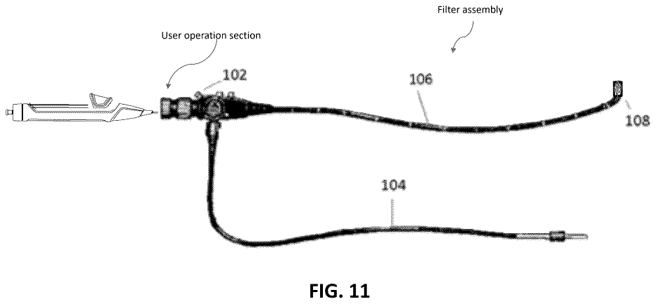

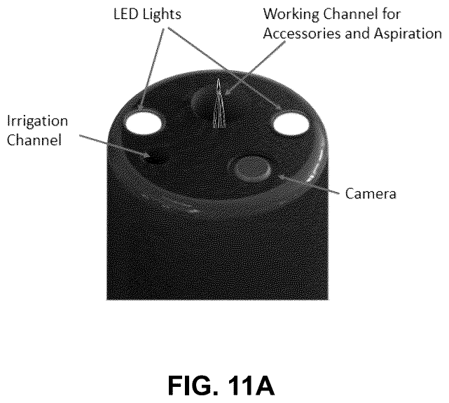

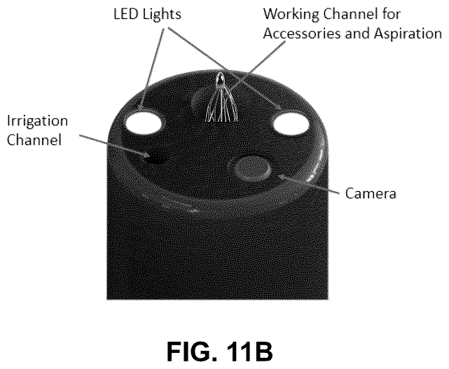

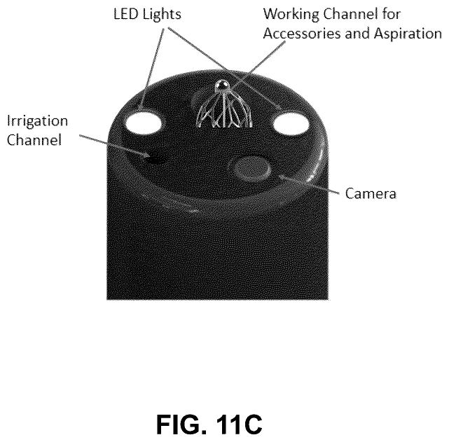

In some embodiments, the filter element of the present disclosure can be made out of pliable material, including metal, plastic, or a combination thereof. The filter element of the filtration device can be deployed inside the distal opening of the working channel, for example as illustrated in FIGS. 11, 11A-C, 12, 12A-C. FIG. 11 in particular is a schematic diagram showing one embodiment of the filtration device of FIG. 7 integrated with the endoscope of FIG. 1 where the filter element stays inside distal opening of the working channel of the endoscope. The collapsed, semi-open, and deployed configurations of the filter element are shown in the enlarged view of the distal section of the endoscope of FIG. 11 in stages A, B, and C of deployment in FIGS. 11A, 11B, and 11C respectively. Because of the pliable nature of the filter element, it pushes against the wall of the distal opening of the working channel to provide a seal and thereby providing filtration to fluid suctioned therethrough.

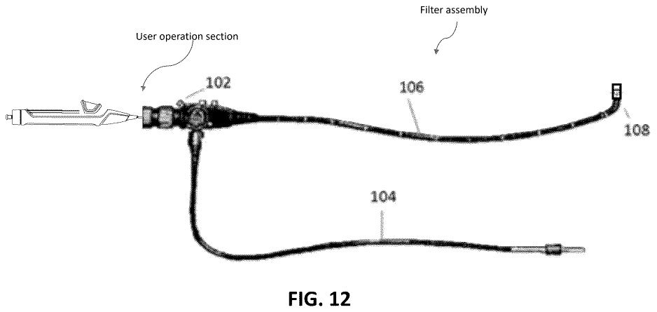

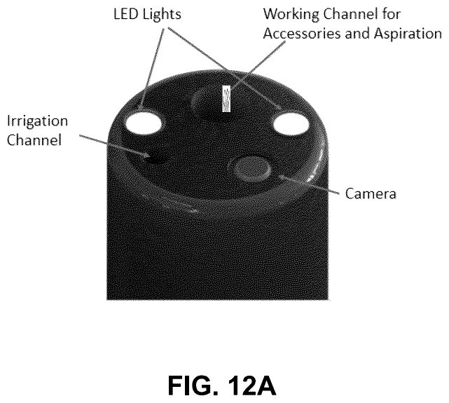

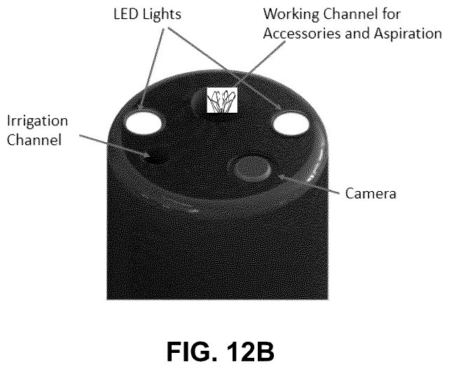

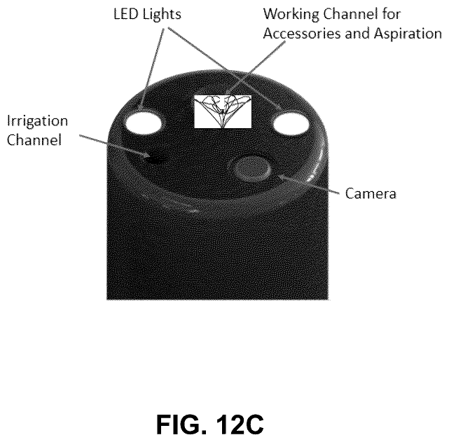

FIG. 12 is a schematic diagram showing one embodiment of the filtration device of FIG. 9 integrated with the endoscope of FIG. 1 where the filter element stays inside distal opening of the working channel of the endoscope. The collapsed, semi-open, and deployed configurations of the filter element are shown in the enlarged view of the distal section of the endoscope of FIG. 12 in stages A, B, and C of deployment in FIGS. 12A, 12B, and 12C respectively. Because of the pliable nature of the filter element, it pushes against the wall of the distal opening of the working channel to provide a seal and thereby providing filtration to fluid suctioned therethrough.

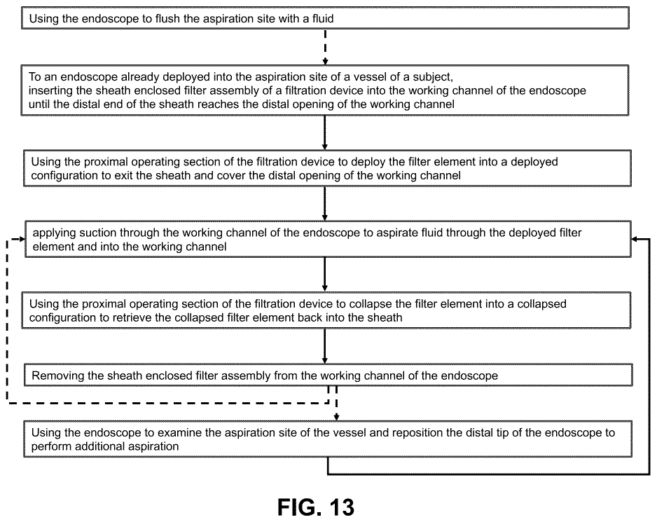

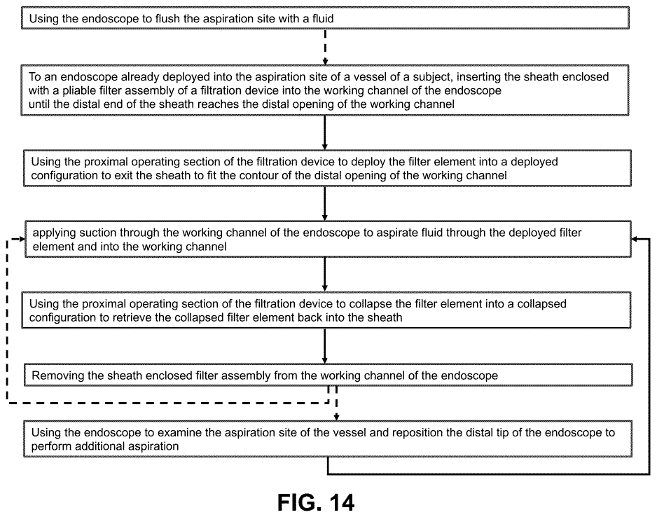

FIG. 13 illustrates the steps in a method of using the filtration device disclosed herein. FIG. 14 illustrates the steps in a method of using the filtration device disclosed herein, where the filter element stays inside the distal opening of the working channel of the endoscope as illustrated in FIGS. 11, 11A-C, 12, and 12A-C.

Specifically, referring to FIG. 13, the method of using the filtration device disclosed herein comprises the steps of inserting the sheath enclosed with the filter assembly of the filtration device into the working channel of the endoscope until the distal end of the sheath reaches the distal opening of the working channel, using the proximal operating section of the filtration device to deploy the filter element into the deployed configuration to exit the sheath and cover the distal opening of the working channel, applying suction through the working channel of the endoscope to aspirate fluid through the deployed filter element and into the working channel, using the proximal operating section of the filtration device to collapse the filter element into a collapsed configuration to retrieve the collapsed filter element back into the sheath, and removing the sheath enclosed filter assembly from the working channel of the endoscope. The endoscope can then be used to examine the aspiration site of the vessel and the distal tip of the endoscope can be repositioned to perform additional aspiration. In some embodiments, the endoscope can be used to flush the aspiration site with a fluid before the aspiration using the filtration element disclosed herein. In some embodiments, after the first round of aspiration, the endoscope can be used to further flush the aspiration site to further rinse and clean the aspiration site followed by an additional aspiration to remove the rinsing fluid.

The aspiration process using the filtration element disclosed herein can be used to clear fluid for better visualization in colonoscopy. In some embodiments, the aspiration process using the filtration element disclosed herein can be performed to clear liquid blood from blood clots in Upper GI bleeding cases. In some embodiment, the aspiration using the filtration element disclosed herein can be performed to hold and release a large polyp which is unable to be aspirated out from the working channel of colonoscope in place of a Roth net. In some embodiment, the aspiration using the filtration element disclosed herein can be performed to help with removal of food bolus or foreign body obstruction of esophagus. The filtration device disclosed herein can be used with an endoscope for an upper endoscopy, a lower endoscopy or a colonoscopy, a bronchoscopy, a thoracoscopy, a laparoscopy, or a video endoscopy.

Specifically, referring to FIG. 14, the method of using the filtration device disclosed herein comprises the steps of inserting the sheath enclosed with the filter assembly of the filtration device into the working channel of the endoscope until the distal end of the sheath reaches the distal opening of the working channel, using the proximal operating section of the filtration device to deploy the filter element into the deployed configuration to exit the sheath and comply the contour of the distal opening of the working channel, applying suction through the working channel of the endoscope to aspirate fluid through the deployed filter element and into the working channel, using the proximal operating section of the filtration device to collapse the filter element into a collapsed configuration to retrieve the collapsed filter element back into the sheath, and removing the sheath enclosed filter assembly from the working channel of the endoscope. The endoscope can then be used to examine the aspiration site of the vessel and the distal tip of the endoscope can be repositioned to perform additional aspiration. In some embodiments, the endoscope can be used to flush the aspiration site with a fluid before the aspiration using the filtration element disclosed herein. In some embodiments, after the first round of aspiration, the endoscope can be used to further flush the aspiration site to further rinse and clean the aspiration site followed by an additional aspiration to remove the rinsing fluid.

In the embodiments where the filter element stays inside the distal opening of the working channel, besides preventing debris that is larger than diameter of the distal opening of the working channel from entering the working channel, in the event where debris that is larger than diameter of the distal opening of the working channel blocks the distal opening, it has the added advantage to push the filter element with or without the sheath forward distally to push away the debris to dislodge the clogging. In some instances, If there are debris stuck in the pores of the filter element to prevent it from getting back to the collapsed configuration, retrieval the filter element back into the distal opening become problematic. But if the filter element stays inside the distal opening of the working channel, it will not run into this problem. Having the filter element staying inside the distal opening of the working channel therefore offer an alternative approach to perform effective aspiration during an endoscopic procedure.

There are currently no devices in outpatient clinical settings which specifically address the clinical problem of endoscope clogging^{6,7,8,9}. However, a single-use device, called the Pure-Vu, provides stronger irrigation spray and additional suction during inpatient procedures. This device is an external sleeve which slides over the colonoscope and can be activated using foot pedals¹⁹. Priced at $350, the Pure-Vu is significantly more expensive than other single-use equipment in colonoscopy procedures²⁰. Furthermore, the device must be fitted to the scope before being placed inside the patient, meaning the cost is incurred for every patient regardless of the quality of colon prep. Even if the irrigation is able to clear waste from the colon wall, the suction can still become clogged with the large pieces of solid material that are dislodged as this device has no feature to address filtering. The device is also only approved for inpatient procedures, meaning it cannot be used for outpatient CRC screenings. Complaints about the product, recorded in the FDA MAUDE database, report instances of bowel perforations accredited to device handling issues and lack of training on a colonoscope that has an increased diameter due to the sleeve design²¹. Therefore, technological challenges exist in creating a device that not only filters solid material, but is inexpensive, fits within the endo scope to avoid an increased diameter, and has a simple mechanism that physicians are familiar with so they do not need extensive training.

The EndoSieve or EndoMesh disclosed herein is designed specifically to address the clinical problem of suction clogging. It acts as a filter to protect the suction or working channel from being clogged with solid material, only allowing liquid to enter the channel. Since the EndoSieve or EndoMesh can be inserted through the instrument channel of the colonoscopy at any point in the procedure, physicians are able to enter the colon and assess the prep, then decide if the EndoSieve or EndoMesh is necessary. This means that cost will only be incurred for patients that require use of the EndoSieve or EndoMesh, not every patient. Furthermore, the EndoSieve or EndoMesh is inserted through the suction or working channel similar to other devices like the Roth Net, so physicians will not require additional training to operate it. Its features and operation will be familiar to physicians because they have experience operating devices that are inserted through the suction channel.

The embodiments above are intended to be illustrative and not limiting. Additional embodiments are within the claims. In addition, although the present disclosure has been described with reference to particular embodiments, those skilled in the art will recognize that changes can be made in form and detail without departing from the spirit and scope of the disclosure. Any incorporation by reference of documents above is limited such that no subject matter is incorporated that is contrary to the explicit disclosure herein.

### CITATIONS

What is colorectal cancer? Cdc.gov. Published February 23, 2023. Accessed February 12, 2024. https://www.cdc.gov/cancer/colorectal/basic_info/what-is-colorectal-cancer.htm
Colonoscopy. Cleveland Clinic. Accessed February 12, 2024. https://my.clevelandclinic.org/health/diagnostics/4949-colonoscopy
Clark B T, Rustagi T, Laine L. What level of bowel prep quality requires early repeat colonoscopy: Systematic review and meta-analysis of the impact of preparation quality on adenoma detection rate. Am J Gastroenterol. 2014;109(11):1714-1723. doi:10.1038/ajg.2014.232
Sim JS, Koo JS. Predictors of inadequate bowel preparation and salvage options on colonoscopy. Clin Endosc. 2016; 49(4):346-349. doi:10.5946/ce.2016.094
Voigt J, Mosier M, Gralnek IM. Colonoscopy in poorly prepped colons: a cost effectiveness analysis comparing standard of care to a new cleansing technology. Cost Eff Resour Alloc. 2021;19(1). doi:10.1186/s12962-021-00277-5
Brown, Jason. January 26, 2024, Joline Speck, Isabella Stagg, Suction Clogging During Endoscopic Procedures
Biyyani Sappati, Raja. January 31, 2024, Sophia Alexander, Lauren Freeman, Joline Speck, Isabella Stagg, Kendall Via, Need for Filtration During Colonoscopic Procedures
Dunlap, Deloria. January 22, 2024. Isabella Stagg, Equipment Issues and Complications During Endoscopies
Shahnavaz, Nikrad. January 31, 2024. Isabella Stagg, Kendall Via, Issues During Endoscopic Procedures
Sauer, Cary. February 1, 2024. Joline Speck, Polyp Removal and Common Challenges During Colonoscopy Procedures
Dhere, Tanvi. January 26, 2024, Lauren Freeman, Kendall Via, Colonoscopies and Roth Net Weaknesses
Cassani, Lisa. January 30, 2024, Joline Speck, Kendall Via, Endoscopic Procedures and Limitations of the Roth Net
Khare, Anshika. January 31, 2024. Sophia Alexander, Lauren Freeman, Approach to Polyps and Solid Material Encountered During Colonoscopy
Messallam, Ahmed. January 25, 2024. Sophia Alexander, Joline Speck, Processes of Polyp Removal and Clearing Suction Clogging
Patel, Akit. February 1, 2024. Lauren Freeman, Joline Speck, Roth Net Shortcomings and Polyp Removal
Ballard, Gwen. January 20, 2024. Sophia Alexander, Joline Speck, Procedural Delays and Patient Impact
Voigt J, Mosier M, Gralnek IM. Colonoscopy in poorly prepped colons: a cost effectiveness analysis comparing standard of care to a new cleansing technology. Cost Effectiveness and Resource Allocation. 2021/04/29 2021;19(1):25. doi:10.1186/s12962-021-00277-5
00711050: STERIS ROTH NET RETRIEVER, FOREIGN BODY, STANDARD 230CM. esutures.com.
Motusgi.com. Accessed February 12, 2024.
Pure-Vu Brochure. MotusGi. AI. Acessed February 8, 2024. https://www.motusgi.com/site/assets/files/1142/pure-vu_brochure.pdf
Gross SA, Gerson LB, Lewis BS, Ganz RA. A novel device for improving visualization in an inadequately prepared colon. Gastrointest Endosc. 2018;87(3):883-888. doi:10.1016/j.gie.2017.10.028
Nitsan et al., inventor; Jet Prep Ltd., assignee. Body passage cleansing device. U.S Pat. No. 9,333,287B2. Patent application Ser. No. 12/923,796. 2016.
Hassidov et. al., inventor; Motus GI Medical Technologies Ltd, assignee. Colon cleaning system with automatic self-purging features. U.S. Pat. No. 11,904,085B2. Patent application Ser. No. 17/516,787. 2024.
Ranallo et al., inventor; United States Endoscopy Group, Inc., assignee. Retrieval device. U.S. Pat. No. 11,871,957B2. Patent application Ser. No. 16/846,581. 2024.
Ahlawat R, Hoilat GJ, Ross AB. Esophagogastroduodenoscopy. StatPearls Publishing; 2023.
Brown, Jason. January 26, 2024, Joline Speck, Isabella Stagg, Suction Clogging During Endoscopic Procedures
Sim JS, Koo JS. Predictors of inadequate bowel preparation and salvage options on colonoscopy. Clin Endosc. 2016;49(4):346-349. doi:10.5946/ce.2016.094
Shahnavaz, Nikrad. January 31, 2024. Isabella Stagg, Kendall Via, Issues During Endoscopic Procedures
Dhere, Tanvi. Jan. 26, 2024, Lauren Freeman, Kendall Via, Colonoscopies and Roth Net Weaknesses

What is claimed is:

1. A filtration device suitable to fit into a working channel of an endoscope, comprising:
a sheath having a proximal end, a distal end, and a distal opening,
a proximal operating section connected with the proximal end of the sheath, and
a filter assembly enclosed inside the sheath comprising a proximal end connected by a middle section to a filter element positioned close to the distal opening of the sheath,
wherein the proximal operating section of the filtration device is operably connected to the proximal end of the filter assembly and capable of deploying the filter element of the filter assembly to exit the distal opening of the sheath to assume a deployed configuration,
wherein the deployed configuration of the filter element has an outer diameter that is greater than or equal to the diameter of a distal opening of a working channel of an endoscope to provide filtration of the fluid that is suctioned through the working channel of the endoscope, and
wherein in the deployed configuration, the filter element comprises pores that are less than 2.5 mm in diameter.

2. The filtration device of claim 1, wherein the filter element comprises a three-dimensional cage in the deployed configuration that has a diameter of at least 3 mm.

3. The filtration device of claim 1, wherein the filter element comprises an inverted umbrella shaped filter element in the deployed configuration that has a diameter of at least 3 mm.

4. The filtration device of claim 1, wherein the endoscope is used for an upper endoscopy, a lower endoscopy or a colonoscopy, a bronchoscopy, a thoracoscopy, a laparoscopy, or a video endoscopy.

5. The filtration device of claim 1, wherein the proximal operating section of the filtration device comprises a clip or a biopsy handle to perform the operation of the filter assembly.

6. The filtration device of claim 1, wherein the filter element is made from metal wires.

7. The filtration device of claim 1, wherein the filter element is made from memory metal wires.

8. The filtration device of claim 1, wherein the filter element is pliable.

9. The filtration device of claim 1, wherein the pliable filter element fits the contour of the working channel.

10. A method of using a filtration device for aspiration during an endoscopy procedure using an endoscope, wherein the endoscope comprises a working channel that has a distal opening at the distal tip, and
the filtration device comprises a sheath having a proximal end, a distal end, and a distal opening; a proximal operating section connected with the proximal end of the sheath; and a filter assembly enclosed inside the sheath comprising a proximal end connected by a middle section to a filter element positioned close to the distal opening of the sheath, wherein the proximal operating section of the filtration device is operably connected to the proximal end of the filter assembly and capable of deploying the filter element of the filter assembly to exit the distal opening of the sheath to assume a deployed configuration, wherein the deployed configuration of the filter element has an outer diameter that is greater than or equal to the diameter of a working channel of an endoscope,
the method comprising:
inserting the sheath enclosed with the filter assembly of the filtration device into the working channel of the endoscope until the distal end of the sheath reaches the distal opening of the working channel,
using the proximal operating section of the filtration device to deploy the filter element into the deployed configuration to exit the sheath and cover the distal opening of the working channel, and
applying suction through the working channel of the endoscope to aspirate fluid through the deployed filter element and into the working channel.

11. The method of claim 10, further comprising using the proximal operating section of the filtration device to collapse the filter element into a collapsed configuration to retrieve the collapsed filter element back into the sheath and removing the sheath enclosed filter assembly from the working channel of the endoscope.

12. The method of claim 10, further comprising using the endoscope to examine the aspiration site of the vessel and reposition the distal tip of the endoscope to perform additional aspiration.

13. The method of claim 10, further comprising using the endoscope to flush the aspiration site with a fluid before the aspiration.

14. The method of claim 10, further comprising using the endoscope to flush the aspiration site with a fluid after the aspiration to further rinse the aspiration site followed by an additional aspiration.

15. The method of claim 10, wherein the aspiration is performed to clear fluid for better visualization in colonoscopy.

16. The method of claim 10, wherein the aspiration is performed to clear liquid blood from blood clots in Upper GI bleeding cases.

17. The method of claim 10, wherein the aspiration is performed to hold and release a large polyp which is unable to be aspirated out from the working channel of colonoscope in place of a Roth net to help with removal of food bolus or foreign body obstruction of esophagus.

18. The method of claim 10, wherein the method is used for an upper endoscopy, a lower endoscopy or a colonoscopy, a bronchoscopy, a thoracoscopy, a laparoscopy, or a video endoscopy.

19. A filtration device suitable to fit into a working channel of an endoscope, comprising:
a sheath having a proximal end, a distal end, and a distal opening,
a proximal operating section connected with the proximal end of the sheath, and
a pliable filter assembly enclosed inside the sheath comprising a proximal end connected by a middle section to a filter element positioned close to the distal opening of the sheath,
wherein the proximal operating section of the filtration device is operably connected to the proximal end of the filter assembly and capable of deploying the filter element of the filter assembly to exit the distal opening of the sheath to assume a deployed configuration,
wherein the deployed configuration of the filter element has an outer diameter that is greater than or equal to the diameter of a distal opening of a working channel of an endoscope to provide filtration of the fluid that is suctioned through the working channel of the endoscope, and
wherein in the deployed configuration, the filter element comprises pores that are less than 2.5 mm in diameter.

20. A method of using the filtration device of claim 19 for aspiration during an endoscopy using an endoscope, wherein the endoscope comprises a working channel that has a distal opening at the distal tip, the method comprising:
inserting the sheath enclosed with the pliable filter assembly of the filtration device into the working channel of the endoscope until the distal end of the sheath reaches the distal opening of the working channel,
using the proximal operating section of the filtration device to deploy the pliable filter element into the deployed configuration to exit the sheath to fit the contour of the distal opening of the working channel,
applying suction through the working channel of the endoscope to aspirate fluid through the deployed filter element and into working channel.
